(19) United States
(12) Patent Application Publication
(10) Pub. No.: US 2008/0064889 A1
(43) Pub. Date: Mar. 13, 2008

(54) Process for Recovering Oils Entrained by Gums Separated During a Degumming Process for Oils made from Oilseeds and Parts thereof, Lecithins Obtained by said Process, and Solvent-Extracted Meals Comprising a Substantially Higher Protein Content

(75) Inventor: Luis E. Palacios, Ciudad De Buenos Aires (AR)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Luis Eduardo Palacios, Ciudad De Buenos Aires (AR)

(21) Appl. No.: 11/839,374

(22) Filed: Aug. 15, 2007

(30) Foreign Application Priority Data
Aug. 15, 2006 (AR) P20060103559

(51) Int. Cl. C07F 9/10; C07C 51/42; C11B 3/02

(52) U.S. Cl. 554/80; 554/175; 554/206

(57) ABSTRACT

A process for recovering oil entrained by the gums separated during an oilseed oil degumming process, comprising the stages of: a) making a countercurrent liquid/liquid solvent extraction of gums separated during degumming of oilseed oil; b) separating solvents from oil by in vacuo distillation in at least three steps; c) recovering the solvent from the extracted gums using a desolventizing equipment; d) adjusting the grain size of the dry gums (lecithins) thus obtained; and e) recovering the solvent from steps b) and c) in the system condensers and rectifying it in a standard rectifying column for reutilization in the process. Preferently, oilseeds or parts of oilseeds are selected from soybean, sunflower, and peanut. The solvent is selected from linear or branched C3-C6 ketones, linear or branched C3-C6 esters, linear or branched C3-C6 alcohols, linear or branched C3-C6 diols, and mixtures thereof. Preferently, the solvent is acetone. The invention also refers to dry particulate lecithins obtained by said process, useful as emulsifiers or dietary supplements, and to extraction meals comprising a substantially higher protein content as a result of not adding the product obtained from the process of the present invention.

### CROSS-REFERENCE TO RELATED APPLICATION

The priority benefit of Argentina patent application P20060103559 filed Aug. 15, 2006, the entire disclosure of which is incorporated by reference herein, is claimed.

### FIELD OF THE INVENTION

The present invention is directed to processes for separating gums (phosphatides) from edible oils, also known as degumming processes. Particularly, the invention refers to a process for recovering oils entrained by gums separated by a degumming process by reason of inefficiencies inherent to the process which cannot not be improved using the currently available technology.

### BACKGROUND OF THE INVENTION

As a part of the process for obtaining vegetable oils from seeds or parts of oilseeds in general and, particularly, from soybean, sunflower, and peanut, by solvent extraction, it is necessary to separate some of the phosphorus present in the thus obtained crude oil in order meet marketing standards and contribute to improve its preservation features.

Separation is achieved by hydrating the crude oils obtained through a solvent-extraction process and then separating the phosphatides present therein using centrifugal separators.

These phosphatides separated from the treated crude oil contain an average of about 33% of entrained crude oil on a dry basis.

In the particular case of soybean, for example, depending on the percentage of oil extracted with hexane solvent from the seeds and the phosphatide content present in said oil, the dry weight of said phosphatides plus the entrained oil may amount to up to about 7.1 kg per ton of processed seed. It is very likely that this FIGURE will increase in the future if the present trend of growing phosphorus content in crude oils continues.

In the prior art, phosphatides as well as entrained oils are added to extracted meals, thereby increasing their oil content and diluting protein content as compared to dry basis.

As used herein, the term “gums” makes reference to the phosphatides separated from the extracted crude oils.

It is therefore important and necessary to achieve a viable process allowing for the recovery of oils entrained by gums obtained in the degumming process of solvent-extracted oilseed oils, in order to improve the efficiency of vegetable oil extraction.

### BRIEF DESCRIPTION OF THE DRAWINGS

FIG. 1 shows a flow diagram of a preferred embodiment of the oil recovery process from gums separated during the degumming process of solvent-extracted oilseed oils.

### SUMMARY OF THE INVENTION

The present invention is directed to a process for recovering oil entrained by the gums removed during vegetable oil degumming processes, preferently from oilseed oils, comprising the stages of:
a) making a countercurrent liquid/liquid solvent extraction of the gums separated during the degumming process of oilseed oils;
b) separating the solvent from the oil by in vacuo distillation in at least three steps;
c) recovering the solvent from the extracted gums using desolventizing equipment;
d) adjusting the grain size of the dry gums (lecithins) thus obtained; and
e) recovering the solvent from steps b) and c) in the system condensers and rectifying it in a standard rectifying column for subsequent recycling into the process.

Preferently, the oilseeds or parts thereof are selected from soybean, sunflower, and peanut.

The solvent is selected from linear or branched C3-C6 ketones, linear or branched C3-C6 esters, linear or branched C3-C6 alcohols, linear or branched C3-C6 diols, and mixtures thereof. Preferently, the solvent is acetone.

The extraction efficiency of process step a) is above from about 90% to about 95% of the oil entrained by the gums, step a) comprising from 3 to 8 stages depending on the required removal efficiency.

The stages of step b) correspond to a first evaporator, a second evaporator and a stripper or final extractor.

The desolventizing equipment of stage c) comprises indirect-vapor heated trays with a countercurrent direct vapor sweep and, preferently, may operate at normal pressure or under moderate vacuum as per design values.

The solvent extraction efficiency of the solvent/oil mixture and desolventizing of the gums (lecithins) allow for a condensation recovery in the order of 98 to 100% of the solvent used.

Steps c) and d) yield high purity dry gums (lecithins) with a water content from about 5% to about 8% w/w depending on their intended use. Step d) comprises processing the dry gums (lecithins) from step c) in a mill for adjusting its grain size to the required standards for the indended use.

Another object of the present invention comprises dry particulate lecithins obtained by the above-described process, for use as emulsifiers or dietary supplements.

Yet another object of the present invention, is a method to obtain extraction meals comprising a substantially higher protein content as a result of not adding to said meals the product obtained according to the process of the present invention.

### DETAILED DESCRIPTION OF THE INVENTION

In order to contribute to a better understanding of the description of the invention, the terms “about”, “in the order of”, “approximately”, or similar, as used throughout the specification and in the claims, intend to mean that the numeric values involved are close to the threshold values as specifically mentioned and within a range of values determined and comprised by ±20% of said numeric value, preferently ±10% of said value and, even more preferently, ±5% of said value. Ranges are established according to the measurement method and the confidence limits used in the corresponding tests.

As used in the present description, the term “substantially” means that the form, circumstance, size, extent or any other feature described by said term, is as similar as may be desired to said form, circumstance, size, extent or any other feature which is precisely and unequivocally known. In fact, this term is intended to mean that what is described is equivalent for the purposes of the invention or is similar to a specific reference made in the description but not necessarily exactly the same.

The present invention is directed to a process for the recovery in the order of at least from 90 to 95% of oil entrained by gums during the degumming process, thereby increasing the efficiency of the current process of solvent extraction of oils.

The gums are a result of degumming vegetable oils. Preferently, vegetable oils are obtained from oilseeds or parts of oilseeds, particularly selected from soybean, sunflower, and peanut.

After a hydration stage, the oil is centrifuged to separate the gums, which are carried from the centrifuges to a recovery facility that operates independently from the conventional solvent extraction line.

Gums are mixed and extracted countercurrently to the chosen solvent, in a series of liquid/liquid extractors equipped with stirrers in order to achieve an increased extraction efficiency of from about 90 to about 95% of oil content in the gums.

The number of stages depends on the capacity and design conditions of the plant, but may vary from 3 to 8 steps, to achieve separation efficiencies at the previously indicated levels.

The process of recovering oils from gums removed by the degumming process according to the present invention uses a solvent selected from linear or branched C3-C6 ketones, linear or branched C3-C6 esters, linear or branched C3-C6 alcohols, linear or branched C3-C6 diols, and mixtures thereof.

Preferently, the solvent employed in the oil recovery process from gums removed during oilseed oil degumming processes of the present invention is acetone (dimethyl ketone).

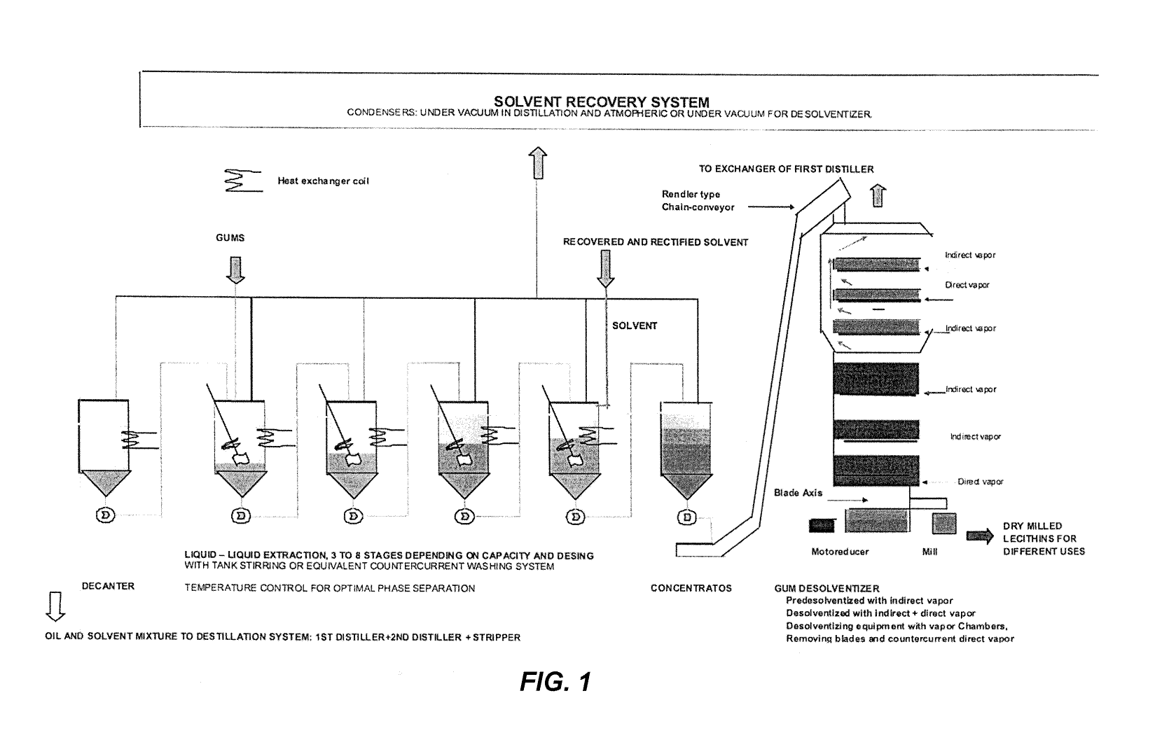

As shown in FIG. 1, the separated oils and gums are concentrated in two decanting end containers: a first decanter and a final concentrator.

The oil is sent to a distillation system comprising at least three stages: a first and second distiller and a final stripper, operating in vacuo under condensation conditions to recover the solvent employed in the process.

Thus, the acetone/oil mixture is pumped into a three-stage distillation system operating in vacuo which separates and recovers by condensation from about 98 to about 100% of the acetone fed in admixture with the crude oil.

The crude oil thus obtained is stored in tanks for commercialization together with the oil directly extracted from seeds.

The extracted gums that may contain from about 15 to about 30% acetone are transported by a chain-conveyor (Redler type) or pumped into a desolventizer where the solvent is recovered by heating and direct vapor exchange to obtain dry lecithins.

In fact, the extracted gums are introduced into a desolventizer, preferently comprised by vapor-heated trays equipped with stirring blades and load control valves for the different levels.

In the first stage, designated pre-desolventization, heat transfer is only indirect and causes evaporation of part of the solvent.

In the second stage, desolventization is completed by maintaining indirect heating but adding direct vapor from a chamber located at the bottom of the equipment which generates a countercurrent sweep through the mass of solids circulating on the perforated surfaces of the desolventizing trays and removing any residual solvent.

The solvent used, preferently acetone, is recovered in distillation condensers and the recovery system in the desolventizer, and subsequently reused in the process by recycling it through a rectification column that uses technology well known in the petrochemical industry.

Finally, the solvent-free dry gums (lecithins) used in the process are milled in order to adjust them to the required grain size for the intended use.

By this process high purity dry gums (lecithins) may be obtained with a water content from about 5% to about 8% w/w according to the intended use.

These lecithins may be used in different industries as natural emulsifiers that are very effective for foodstuff or may be re-added to extraction meals, as they are a natural component thereof.

If the intended use so requires it, the lecithins may be also bleached and filtered in a further stage. It is possible to achieve purities acceptable for dietary supplements.

All system effluents are completely safe since both the small volumes of air that may be entrained in the liquid gums, as well as the reduced flow of water that is not recycled into the process, are treated with high throughput recovery systems of proven reliability and operational safety before discharge.

The amount of acetone used in the process, for example, is less than about 1 dm³ per ton of processed gums. The oil to be recovered per ton of dry gums separated in the centrifuge for the particular case of soybean is of about 330 kg.

All facilities are completely tight. Equipment, engines, handling tools and control facilities are explosion-proof and meet the most rigorous international standards, such as NFPA.

It is also possible to use batch extraction processes, i.e., discontinuous or other known liquid/liquid countercurrent extraction systems for separating oil and gums, without this adjustment being a departure from the scope of the present invention.

Accessorily, a further object of the process of the present invention is to recover dry lecithins that are useful as emulsifiers and, if sufficiently refined, can be used as dietary supplements.

Still another object of the present invention is to substantially improve the protein content of extraction meals, by not adding the product obtained during oil degumming, as was the case in the prior art.

Financial Benefits of the Process

The differential value of recovered products as compared to the state of the art is shown in the accompanying tables, both for the particular case of Argentina as well as for manufacturing industries in other parts of the world.

The process permits the recovery of up to about 2.4 kg of crude oil per ton of processed soybean seeds, depending on the oil content of the seeds and its phosphorus content.

It also produces up to about 5 kg of high purity dry lecithin per ton of processed seed, which may be commercialized as an emulsifier for different industrial applications.

Further, the process produces and increase of more than about 0.5% in the protein concentration of high- and low-protein extraction meals, thus meeting standard market requirements. Consequently, better prices are obtained by avoiding quality discounts. It is also possible to achieve the state of the art protein content in meals by replacing the removed products with a higher water content, thus complying with all the required specifications.

Although the value of the recovered products is much higher than that of their equivalent prior-art meals, only the value of recovered oils has been taken into consideration in the table below.

Even disregarding the differential value of the dry lecithin obtained with the invention, the process will also provide an incremental added value to the manufacturing industry which—though depending on the market value of soybean milling products—would be increased in at least approximately U$S 0.47 per ton of processed soybean seeds in Argentina (calculated including current withholdings on exports), and in the order of U$S 0.61 per ton for the rest of the world, using current market values as a reference.

World soybean production intended for the manufacturing industry is above 200 million tons per year and is permanently increasing.

This process will allow for a minimum additional income of 100 million dollars per year for the soybean transformation industry into crude oils and protein meals, of which about 20% will remain in Argentina, with the current market values as a reference.

### EXAMPLE 1

### Soybean with Acetone as Solvent

The following Tables A and B summarize the advantages of an exemplary process of the invention according to the corresponding mass balance.

TABLE A
ARGENTINA (CONSIDERING TAX WITHHOLDINGS
ON EXPORTS).
SAVINGS RESULTING FROM RECOVERY OF OIL FROM GUMS
Analysis for 1 ton of ground SOYBEAN seed

| | | |
|---|---|---|
| Soybean oil FOB | U$S/Ton | 500 |
| Soybean oil FAS Argentina (20% withholding) | U$S/Ton | 400 |
| Soybean meal FOB | U$S/Ton | 170 |
| Soybean meal FAS Argentina (20% withholding) | U$S/Ton | 136 |
| Value of pure lecithin powder FAS (20% withholding) | U$S/Ton | 136 |
| Crude oil versus soybean meal | U$S/Ton | 264 |

| Item | Detail | Unit | Amount |
|---|---|---|---|
| 1 | Yield % of gum-free crude oil | % | 18.50 |
| 2 | Yield % of gum-free crude oil | Kgs | 185.00 |
| 3 | Contribution of oily gums w/oil | % | 3.76 |
| 4 | Yield % of crude oil with gums | % | 19.2 |
| 5 | Oil per ton of seed | Kgs | 192.2 |
| 6 | Phosphorus content in crude oil without | ppm | 1100 |
| | degumming | | |
| 7 | Phosphorus content in degummed crude | ppm | 200 |
| 8 | Phosphorus difference | ppm | 900 |
| 9 | Conversion factor | | 28 |
| 10 | Insolubles/Total dry oily gums ratio | | 0.67 |
| 11 | Dry deoiled phosphatides | % | 2.52 |
| 12 | Dry deoiled phosphatides | Kgs | 4.84 |
| 13 | Dry oily phosphatides | % | 3.76 |
| 14 | Dry oily phosphatides | Kgs | 7.23 |
| 15 | Phosphatides-entrained oil | % | 1.24 |
| 16 | Phosphatides-entrained oil | Kgs | 2.39 |
| 17 | Phosphatides-entrained oil | U$S/Ton | 0.63 |
| 18 | Yield % of extraction with acetone | % | 90 |
| 19 | Recovered oil | U$S/Ton | 0.57 |
| 20 | Dry lecithins (5% water) | Kgs | 5.10 |
| 21 | Dry lecithins (5% water) - Meal value | U$S/Ton | 0.69 |
| 22 | Incremental operative cost per ton of | U$S/Ton | 0.10 |
| | ground seed | | |
| 23 | Savings - Only for recovered oil | U$S/Ton | 0.47 |

TABLE B
OTHER COUNTRIES (WITHOUT WITHHOLDINGS).
SAVINGS RESULTING FROM RECOVERY OF OIL FROM GUMS
Analysis for 1 ton of ground soybean seed

| | | |
|---|---|---|
| Soybean oil FOB | U$S/Ton | 500 |
| Soybean meal FOB | U$S/Ton | 170 |
| Value of pure lecithin powder FOB | U$S/Ton | 170 |
| Crude oil versus soybean meal | U$S/Ton | 330 |

| Item | Detail | Unit | Amount |
|---|---|---|---|
| 1 | Yield % of gum-free crude oil | % | 18.5 |
| 2 | Yield % of gum-free crude oil | Kgs | 185.0 |
| 3 | Contribution of oily gums w/oil | % | 3.76 |
| 4 | Yield % of crude oil with gums | % | 19.2 |
| 5 | Phosphorus content in non-degummed crude | Kgs | 192.2 |
| 6 | Phosphorus content in non-degummed crude | ppm | 1100 |
| 7 | Phosphorus content in degummed crude | ppm | 200 |
| 8 | Phosphorus difference | ppm | 900 |
| 9 | Conversion factor | | 28 |
| 10 | Insolubles/Total dry oily gums ratio | | 0.67 |
| 11 | Dry deoiled phosphatides | % | 2.52 |
| 12 | Dry deoiled phosphatides | Kgs | 4.84 |
| 13 | Dry oily phosphatides | % | 3.76 |
| 14 | Dry oily phosphatides | Kgs | 7.23 |
| 15 | Phosphatides-entrained oil | % | 1.24 |
| 16 | Phosphatides-entrained oil | Kgs | 2.39 |
| 17 | Phosphatides-entrained oil | U$S/Ton | 0.79 |
| 18 | Yield % of extraction with acetone | % | 90 |
| 19 | Recovered oil | U$S/Ton | 0.71 |
| 20 | Dry lecithins (5% water) | Kgs | 5.10 |
| 21 | Dry lecithins (5% water) - Meal value | U$S/Ton | 0.87 |
| 22 | Incremental operative cost per ton of | U$S/Ton | 0.10 |
| | ground seed | | |
| 23 | Savings - Only for recovered oil | U$S/Ton | 0.61 |

### EXAMPLE 2

### Sunflower with Acetone as Solvent

The following Tables C and D summarize the results of an exemplary process of the present invention according to the corresponding mass balance.

TABLE C
ARGENTINA (WITH WITHHOLDINGS ON EXPORTS).
SAVINGS RESULTING FROM RECOVERY OF OIL FROM GUMS
Analysis for 1 ton of ground SUNFLOWER seed

| | | |
|---|---|---|
| Sunflower oil FOB | U$S/Ton | 500 |
| Soybean oil FAS Argentina (20% withholding) | U$S/Ton | 464 |
| Soybean meal FOB | U$S/Ton | 170 |
| Soybean meal FAS Argentina (20% withholding) | U$S/Ton | 136 |
| Value of pure lecithin powder FAS (20% | U$S/Ton | 136 |
| withholding) | | |
| Crude oil versus soybean meal | U$S/Ton | 328 |

| Item | Detail | Unit | Amount |
|---|---|---|---|
| 1 | Yield % of gum-free crude oil | % | 10.00 |
| 2 | Yield % of gum-free crude oil | Kgs | 100.00 |
| 3 | Contribution of oily gums w/oil | % | 1.466 |
| 4 | Yield % of crude oil with gums | % | 10.15 |
| 5 | Phosphorus content in non-degummed crude | Kgs | 101.48 |
| 6 | Phosphorus content in non-degummed crude | ppm | 500 |
| 7 | Phosphorus content in degummed crude | ppm | 150 |
| 8 | Phosphorus difference | ppm | 350 |
| 9 | Conversion factor | | 28 |
| 10 | Insolubles/total dry oily gums ratio | | 0.67 |
| 11 | Dry deoiled phosphatides | % | 0.98 |
| 12 | Dry deoiled phosphatides | Kgs | 0.99 |
| 13 | Dry oily phosphatides | % | 1.46 |
| 14 | Dry oily phosphatides | Kgs | 1.48 |
| 15 | Oil entrained in phosphatides | % | 0.48 |
| 16 | Oil entrained in phosphatides | Kgs | 0.49 |
| 17 | Oil entrained in phosphatides | U$S/Ton | 0.16 |
| 18 | Yield % of extraction with acetone | % | 90 |
| 19 | Recovered oil | U$S/Ton | 0.14 |
| 20 | Dry lecithins (5% water) | Kgs | 1.05 |
| 21 | Dry lecithins (5% water) - Meal value | U$S/Ton | 0.14 |
| 22 | Incremental operative cost per ton of | U$S/Ton | 0.03 |
| | ground seed | | |
| 23 | Savings - Only for recovered oil | U$S/Ton | 0.12 |

TABLE D
OTHER COUNTRIES (NO WITHHOLDINGS).
SAVINGS RESULTING FROM RECOVERY OF OIL FROM GUMS
Analysis for 1 ton of ground SUNFLOWER seed

| | | |
|---|---|---|
| Sunflower oil FOB | U$S/Ton | 580 |
| Soybean meal FOB | U$S/Ton | 170 |
| Value of pure lecithin powder FOB | U$S/Ton | 170 |
| Crude oil versus soybean meal | U$S/Ton | 410 |

| Item | Detail | Unit | Amount |
|---|---|---|---|
| 1 | Yield % of gum-free crude oil | % | 10.00 |
| 2 | Yield % of gum-free crude oil | Kgs | 100.00 |
| 3 | Contribution of oily gums w/oil | % | 1.46 |
| 4 | Yield % of crude oil with gums | % | 10.15 |
| 5 | Phosphorus content in non-degummed crude | Kgs | 101.48 |
| 6 | Phosphorus content in non-degummed crude | ppm | 500 |
| 7 | Phosphorus content in degummed crude | ppm | 150 |
| 8 | Phosphorus difference | ppm | 350 |
| 9 | Conversion factor | | 28 |
| 10 | Insolubles/Total dry oily gums ratio | | 0.67 |
| 11 | Dry deoiled phosphatides | % | 0.98 |
| 12 | Dry deoiled phosphatides | Kgs | 0.99 |
| 13 | Dry oily phosphatides | % | 1.46 |
| 14 | Dry oily phosphatides | Kgs | 1.48 |
| 15 | Oil entrained in phosphatides | % | 0.48 |
| 16 | Oil entrained in phosphatides | Kgs | 0.49 |
| 17 | Oil entrained in phosphatides | U$S/Ton | 0.20 |
| 18 | Yield % of extraction with acetone | % | 90 |
| 19 | Recovered oil | U$S/Ton | 0.18 |
| 20 | Dry lecithins (5% water) | Kgs | 1.05 |
| 21 | Dry lecithins (5% water) - Meal value | U$S/Ton | 0.18 |
| 22 | Incremental operative cost per ton of | U$S/Ton | 0.03 |
| | ground seed | | |
| 23 | Savings - Only for recovered oil | U$S/Ton | 0.16 |

### INDUSTRIAL APPLICATION OF THE INVENTION

The present invention provides applications in the food industry, specially in the sector involved in solvent extraction of oil from oilseeds, as well as related sectors which are greatly benefited from a recovery process of oil entrained within the gums separated during oil degumming processes in oilseeds, and also from lecithins and extraction meals comprising a substantially higher protein content, thereby providing an important advantage as compared to the prior art.

Final Considerations

Those skilled in the art will readily envision different modifications and variations of the process for recovering oil entrained by gums separated during oilseed oil degumming processes, as well as dry gums (lecithins) and extraction meals comprising a substantially higher protein content, such as described in the present invention, without departing from the scope and spirit of the invention. Although the invention has been described with reference to preferred embodiments, it is to be understood that the invention as claimed should not be limited to such specific embodiments. Indeed, different modifications of the described modes of carrying out the invention, which are obvious to those skilled in the art, are intended to be included within the scope of the following claims.

The claims are a part of the description of the subject matter of the present patent application.

1. A process for recovering oil entrained by gums separated during degumming of oil made from oilseeds or parts thereof, said process comprising the stages of:
a) making a countercurrent liquid/liquid solvent extraction of gums separated during degumming of oil made from oilseeds or parts thereof;
b) separating solvent from oil by in vacuo distillation in at least three steps;
c) recovering the solvent from the extracted gums using desolventizing equipment;
d) adjusting the grain size of the dry gums (lecithins) thus obtained; and
e) recovering the solvent from steps b) and c) in the system condensers and rectifying said solvent in a standard rectifying column for further recycling into the process.

2. The process according to claim 1, wherein the oilseeds are selected from soybean, sunflower, and peanut.

3. The process according to claim 1, wherein the solvent is selected from linear or branched C3-C6 ketones, linear or branched C3-C6 esters, linear or branched C3-C6 alcohols, linear or branched C3-C6 diols, and mixtures thereof.

4. The process according to claim 2, wherein the solvent is acetone.

5. The process according to claim 1, wherein the extraction efficiency of step a) is higher than from about 90% to about 95% of oil entrained by the gums.

6. The process according to claim 5, wherein sep a) is carried out in 3 to 8 stages according to the required removal efficiency.

7. The process according to claim 1, wherein the stages of step b) correspond to a first evaporator, a second evaporator and a stripper or final extractor.

8. The process according to claim 1, wherein the desolventizing equipment of step c) comprises indirect-vapor heated trays with a countercurrent direct vapor sweep

9. The process according to claim 8, wherein the desolventizing equipment may operate under normal pressure or under moderate vacuum as per design values.

10. The process according to claim 1, wherein the solvent extraction efficiency of the solvent/oil mixture and gum (lecithins) desolventizing allows for a recovery by condensation in the order of from 98 to 100% of solvent used.

11. The process according to claim 1, wherein in steps c) and d) high purity dry gums (lecithins) are obtained with a water content from about 5% to about 8% w/w depending on the intended use.

12. The process according to claim 1, wherein step d) comprises processing the dry gums (lecithins) from step c) in a mill in order to adjust their grain size to the required standards for the intended use.

13. Dry particulate lecithins obtained by the process of claim 1.

14. Lecithins according to claim 1, for use as emulsifiers or dietary supplements.

15. Solvent extraction meals, wherein said meals comprise a substantially higher protein content as a result of not adding the product obtained according to the process of claim 1.
